(19) United States
(12) Patent Application Publication
(10) Pub. No.: US 2009/0171395 A1
(43) Pub. Date: Jul. 2, 2009

(54) Dynamic spinal rod system

(76) Inventors: Dong M. Jeon, Draper, UT (US); Patrick D. Moore, West Jordan, UT (US); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 12/317,866

(22) Filed: Dec. 29, 2008

Related U.S. Application Data
(60) Provisional application No. 61/017,320, filed on Dec. 28, 2007.

(51) Int. Cl. A61B 17/70

(52) U.S. Cl. 606/257; 606/246

(57) ABSTRACT

A dynamic spinal rod system for semi-restrained holding of the vertebrae with respect to each other in a corrected position while providing varying degrees of vertebral body freedom of movement. A first or “male” component has an enlarged proximal end with a bio-compatible elastic polymer insert through which an anchoring component, such as a pedicle screw, may be placed for attachment, and a cylindrical distal end. A second or “female” component includes a similar enlarged proximal end with a bio-compatible elastic polymer insert through which an anchoring component may be placed for attachment, but has a distal end with a colleted portion into which the distal end of the first component may be inserted and retained to form a joined system. A third “interconnector” component may be used to attach to the proximal end of either of the first two components.

### CROSS-REFERENCE TO RELATED APPLICATION

This application claims the benefit of U.S. Provisional Application No. 61/017,320, filed Dec. 28, 2007, the disclosure of which is incorporated herein by reference in its entirety.

### TECHNICAL FIELD

The present invention concerns a dynamic implanted spinal rod system for effecting and maintaining a correction of the relative positions of the vertebrae and/or of the static and dynamic forces exerted on the vertebrae, for treating a congenital or acquired deformation of the spine, in particular an idiopathic condition such as kyphoscoliosis, or a post-traumatic, tumorous, infectious, degenerative or other, instability of the spine.

### BACKGROUND

Spinal devices for reducing scoliotic deformations are already known, consisting of components for anchoring in the vertebrae, such as hooks or intrapedicular screws, and of rods or frames fixed to the anchoring components to impose a relative position on the various vertebrae. These rigid or semi-rigid osteosynthesis devices produce a rigidification of the spinal column in the corrected position and are always associated with a bone graft for spinal fusion. Consequently, the fitting of such an osteosynthesis device has the effect of permanently suppressing the natural physiological mobility of the vertebrae. Thus, these known osteosynthesis devices, though they solve to a large extent the problems connected with scoliotic deformation, necessarily result in a handicap for the patient. This handicap is all the more serious because it is permanent and generally affects patients who are young and still growing. These known devices thus constitute an obstacle to the subsequent growth of the spine.

Furthermore, known osteosynthesis devices pose numerous further problems with regard to the positioning and reliability of the anchoring components, which are subjected to high stresses because of the subsequent rigidity, and the timing of the fixing of the rods, plates or frames to the anchoring components, which must be carried out at the same time as the reduction of the deformation. Various semi-rigid osteosynthesis devices have thus been proposed to resolve these drawbacks by preserving a certain elasticity which assists the fusion of the subsequent bone graft and facilitates the positioning of the anchoring components or reduces the stresses transmitted to the anchoring components. For example, French Patent reference FR-A-2 689 750, the disclosure of which is incorporated by reference herein, discloses a proposed osteosynthesis device in which the rods have a flexibility with a high elastic limit. The elasticity thus preserved in the area of the fusion is proposed to assist the healing of the bone graft. Nevertheless, the problems connected with the rigidification of the column persist after fusion with such a device. Similarly, U.S. Pat. No. 4,836,196, the disclosure of which is incorporated by reference herein, describes a spacing device disposed between anchoring components and a rigidification structure, making it possible to reduce the stresses transmitted between the vertebral body and the structure. Similarly, U.S. Pat. No. 4,573,454, the disclosure of which is incorporated by reference herein, describes a device with an extensible structure consisting of a frame in two parts, one of which telescopes into the other, for the purpose of assisting subsequent growth in spite of the rigidification of the spine. Nevertheless, this only partially addresses the problem since the portions of the spine fixed respectively to each of the parts of the structure are themselves rigidified without growth being possible.

In addition, conditions such as adjacent disk disease are a common complication resulting from solid fusion techniques. Adjacent disk disease may result from the additional lever force applied to adjacent disks as a result from the solid fusion of the disk space and the affected vertebra bodies. This condition often requires additional surgeries to correct, which in turn exacerbates further adjacent disk space degeneration. Providing dynamic stabilization to the adjacent vertebral body at the time of the original surgery could reduce the lever force created from the newly created fusion mass.

A new dynamic implanted spinal rod system which preserves, at least in part, the natural physiological mobility of the vertebrae while effecting and maintaining a correction of the relative positions of the vertebrae with, or without the use of a graft/cage/spacer, or as, as not, an adjunct to fusion would thus be an improvement in the art.

### SUMMARY

In one embodiment, systems in accordance with the present invention provide a dynamic means for a semi-restrained holding of the vertebrae with respect to each other in the corrected position while providing varying degrees of vertebral body freedom of movement. A first or “male” component has an enlarged proximal end with a bio-compatible elastic polymer insert through which an anchoring component, such as a pedicle screw may be placed for attachment, and a cylindrical distal end. A second or “female” component includes a similar enlarged proximal end with a bio-compatible elastic polymer insert through which an anchoring component may be placed for attachment, but has a distal end with a colleted portion into which the distal end of the first component may be inserted and retained to form a joined system. A third “interconnector” component may be used to attach to the proximal end of either of the first two components.

The orientation/placement of the device provides a means of holding the vertebrae in the corrected position against natural deforming forces and reducing the forces exerted on the vertebrae while preserving relative amounts of mobility, or as dynamic stabilization as an adjunct to fusion for treating a congenital or acquired deformation of the spine, in particular an idiopathic condition such as kypho-scoliosis, or a post-traumatic, tumorous, infectious, degenerative or other instability of the spine. Thus the invention aims to propose a novel category of dynamic implanted spinal orthosis which, unlike known osteosynthesis devices preserves, at least in part, the natural physiological mobility of the vertebrae while effecting and maintaining a correction of the relative positions of the vertebrae with, or without the use of a graft/cage/spacer, or as, or as not, an adjunct to fusion.

### DESCRIPTION OF THE DRAWINGS

It will be appreciated by those of ordinary skill in the art that the elements depicted in the various drawings are not necessarily to scale, but are for illustrative purposes only. The nature of the present invention, as well as other embodiments of the present invention may be more clearly understood by reference to the following detailed description of the invention, to the appended claims, and to the several drawings attached hereto.

FIGS. 1A, 1B and 1C are front, side, and end views of a first embodiment of a “male” component for a dynamic implanted spinal rod system in accordance with the present invention, having a proximal end with a constrained bio-compatible elastic polymer insert and a distal end with a cylindrical portion.

FIGS. 2A, 2B and 2C are front, side, and end views of a “female” component for a dynamic implanted spinal rod system in accordance with the present invention with a proximal end with a constrained bio-compatible elastic polymer insert and distal end including an externally threaded female colleted portion.

FIGS. 3A and 3B depict front and side views of the embodiments of FIGS. 1A through 2C attached to one another to form a dynamic implanted spinal rod system in accordance with the present invention.

FIGS. 4A and 4B are front and side views of a portion of the dynamic spinal rod system of FIGS. 3A through 3C, joined to an interconnector component with an end geometry that interfaces around the top and bottom of end geometry of the proximal end of the “female” component of the dynamic implanted spinal rod system in accordance with the present invention.

### DETAILED DESCRIPTION

For the purposes of promoting an understanding of the principles of the invention, reference will now be made to the embodiment illustrated in the drawings, and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

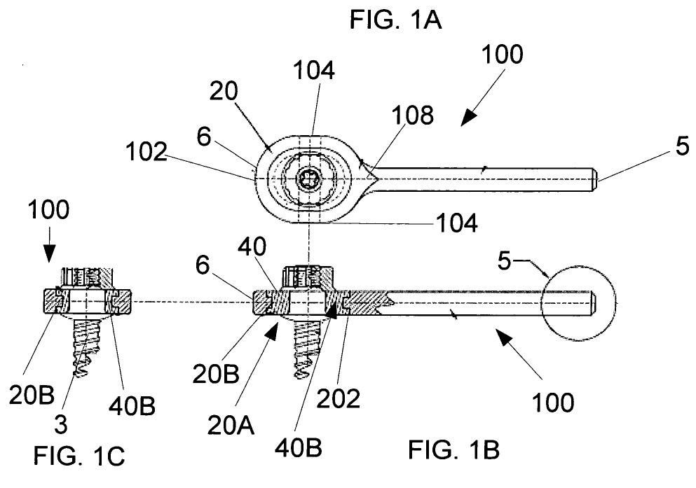

Referring generally to FIGS. 1A, 1B and 1C, there is depicted one embodiment of a “male” rod component 100 for a dynamic implanted spinal rod system which has a proximal end 6 and a distal end 5. Proximal end 6 has an enlarged end 20, formed as a section with opposite flat upper and lower surfaces forming a body curving from a point 102 at distal end 6 to two opposite sidewalls 104 and 106 to an opposite curve that joins at a point 108 directly opposite point 102 on distal end 6. An opening 20A passes from the upper surface to the lower surface through the enlarged end 20 body. The sidewall 202 of opening 20A may follow the shape of the outer sidewall of the enlarged end body, having an oval shape, although other shapes may be used. A ridge 20B is disposed on the inner sidewall 202 and may be formed as a raised section of the sidewall forming a raised wall encircling the entirety of opening 20A. A bio-compatible elastic polymer insert 40 is disposed in opening 20A, and may be secured therein by a counterpart receiving slot 40B that resides on ridge 20B. Insert 40 may include an opening 40A allowing an anchoring component, such as a pedicle screw 3 to pass at least partially therethrough. It will be appreciated that although a pedicle screw is depicted as an anchoring component, any suitable bone anchor, including poly-axial pedicle screws, mono-axial pedicle screws, lamina or pedicle hooks, or any other side loading spinal fusion systems may be used.

Bio-compatible elastic polymer insert 40 may thereby movably restrain the anchoring component 3 fixed to a vertebrae as a holding means and provide a method of attachment to the dynamic spinal rod system. Between proximal end 6 and distal end 5, the component includes cylinder section 1 which is essentially a rod extending from the enlarged end body to distal end 5.

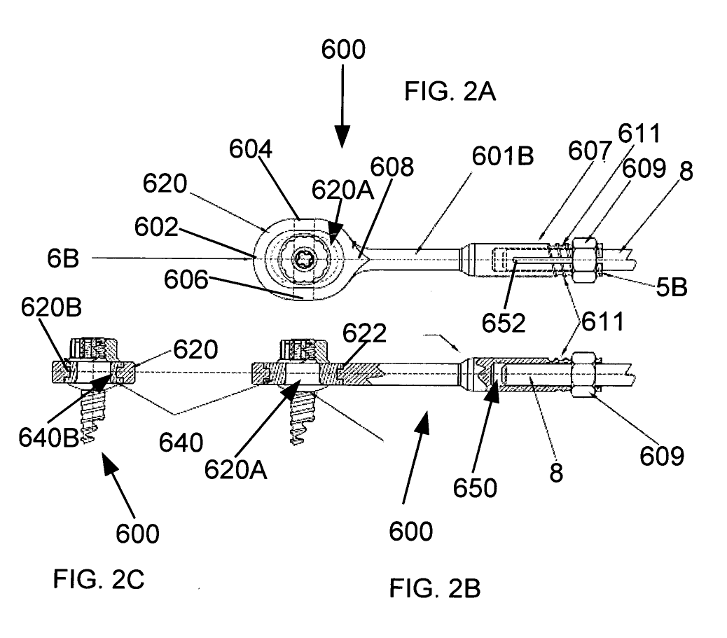

Referring generally to FIGS. 2A, 2B and 2C, there is depicted one embodiment of a “female” rod component 600 for a dynamic implanted spinal rod system which has a proximal end 6B and a distal end 5B. Proximal end 6B has an enlarged end 620, which is similar to enlarged end 20 of male component 100 depicted in FIGS. 1A, 1B, and 1C. Enlarged end 620 has a body with opposite flat upper and lower surfaces and an outer sidewall curving from a point 602 at distal end 6B to two opposite sidewalls 604 and 606 to an opposite curve that joins at a point 608 directly opposite point 602 on distal end 6B. An opening 620A passes from the upper surface to the lower surface through the enlarged end 620 body. The sidewall 622 of opening 620A may follow the shape of the outer sidewall of the enlarged end body, having an oval shape, although other shapes may be used. A ridge 620B is disposed on the inner sidewall 622 and may be formed as a raised section of the sidewall forming a raised wall encircling the entirety of opening 620A. A bio-compatible elastic polymer insert 640; is disposed in opening 620A, and may be secured therein by a counterpart receiving slot 640B that resides on ridge 620B. Insert 640 may include an opening 640A allowing an anchoring component, such as a pedicle screw 3 to pass at least partially therethrough. Bio-compatible elastic polymer insert 640 may thereby movably restrain the anchoring components 3 fixed to a vertebra as a holding means, and provide a method of attachment to the dynamic spinal rod system.

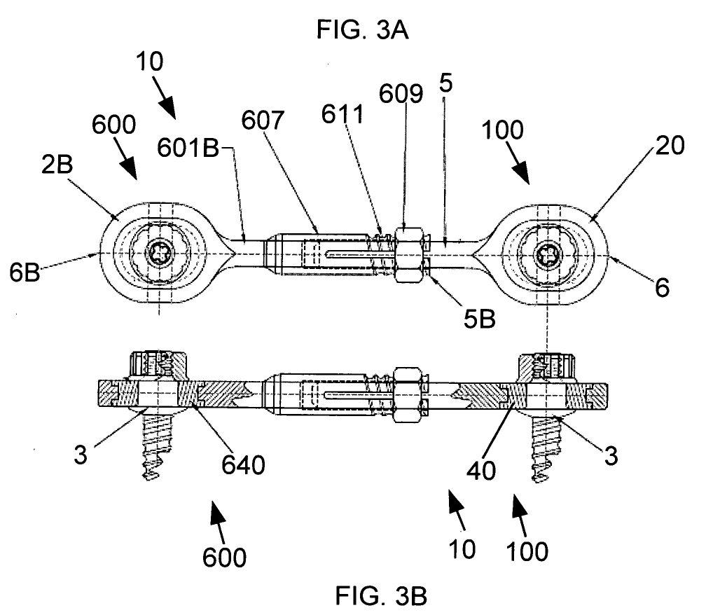

Between proximal end 6B and distal end 5B, female component 600 may have a medial cylinder section 601B which is essentially a rod. Distal to the medial cylinder section 601B is an externally threaded female colleted portion 607. Colleted portion 607 may be formed as a tube 650 having an open bottom at distal end 5B, with one or more slots 652 formed through the sidewall thereof, passing from distal end 5B towards the proximal end 6B. In the depicted embodiments, two slots 652 may be used, but it will be appreciated the number may vary as suitable for a particular usage. A male cylindrical member 8, such as a rod, or the distal end 5 of the embodiment shown in FIGS. 1A, 1B and 1C may be inserted into the tube 650 from distal end 5B. Following insertion of a cylindrical member 8, an internally threaded nut 609 disposed on external threads 611 may be rotated to tighten the collet, retaining the cylindrical member within the colleted portion 607 and securing the relative position of the members. FIGS. 3A and 3B depict one such joining of the embodiments of FIGS. 1A through 2C, with two opposite proximal ends 6 and 6B and two joined distal ends 5 and 5B forming a new medial portion to form a complete dynamic implanted spinal rod system 10, in accordance with the present invention.

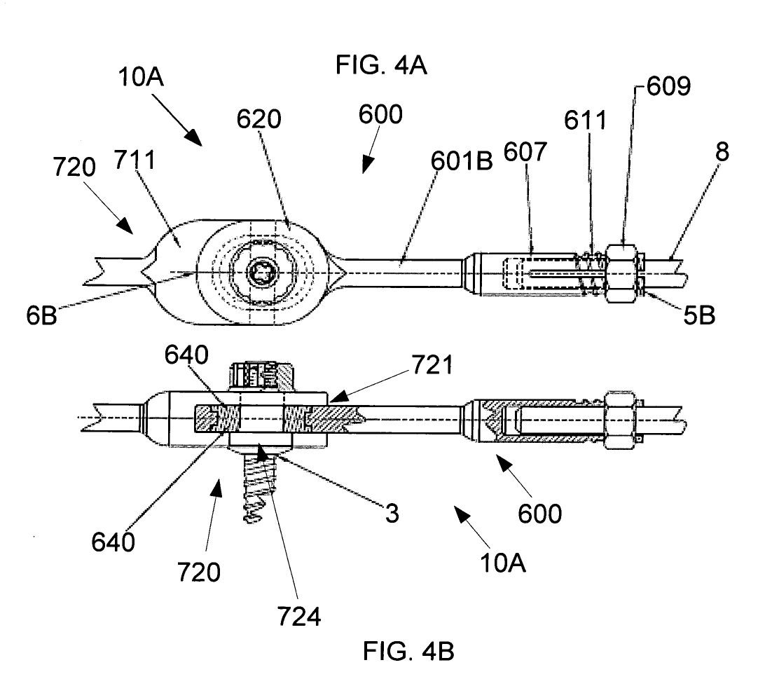

Referring generally to FIGS. 4A and 4B, there is depicted one embodiment of a dynamic implanted spinal rod system 10A which has a female component 600, as depicted in FIGS. 2A, 2B and 2C. Distal end 5B includes female colleted portion 7 in which a male cylindrical rod 8 is restrained as discussed previously herein. An interconnector component 720 includes a distal end 711 which is configured to interface around the top and bottom of the enlarged proximal end 602B of the female component 600. A slot 721 is formed in an enlarged portion of distal end 711 running parallel to the long axis of the component 720. The walls of the slot 721 are configured to retain proximal end 6B therein, residing against the upper and lower surfaces of the enlarged end 602 of the female component. The bio-compatible insert 640 is further restrained by these walls. An anchoring opening 724 passes through the distal end 711, generally perpendicular to the slot 721 to allow an anchoring component, such as a pedicle screw 3, to be placed therethrough, joining the components together. Interconnector component 720 extends from distal end 711 to a proximal end, not depicted, which may be a rod or a dynamic attachment end, similar to the proximal ends of the other components 100 and 600 discussed herein.

Preferred materials for the present invention include stainless steel, titanium, Nitinol, or even PEEK (polyetheretherketone polymer) for the suitable adjustable metallic rod device components for maintaining a correction of the positions of the vertebrae for treating a deformation/disease of the spine, and an elastic polymer insert located either proximally, distally, or both within the metallic rod device for corrected positioning of the vertebral bodies and holding the anchoring component may be formed from a suitable polymer, such as a polycarbonate urethane polymer (PCU). In other embodiments, the insert may be manufactured from a suitable metallic material and restrained movably within the same end geometry, as by residing within a slot or upon a ridge. It will be recognized that any sturdy biocompatible material suitable for the purpose may be used to construct the components and achieve the osteosynthesis and other orthopedic surgical goals of the present invention.

One advantage of a system in accordance with the present invention is that such dynamic spinal rod systems can subsequently be removed, in particular at the end of the period of growth when the risks of a worsening or recurrence of the deformation or instability have disappeared. Such systems may also provide a dynamic spinal rod system which can be fitted with the minimum possible risk of affecting the nervous system, as the dynamic system may be emplaced in a manner similar to a traditional fixed rod system. This additionally reduces the stress on the system by reducing the stress transmitted to the anchoring components fixed to the vertebrae to the lowest possible mechanical stresses, and in particular mechanical stresses of a magnitude which is strictly limited to that necessary for maintaining the correction of the deformation and/or applying the desired forces to the vertebrae.

Such a system allows the characteristics of alignment to be adjusted during fitting, thus enabling deformation of the spinal column to be reduced accurately in three dimensions. Such systems therefore enable scoliosis to be reduced while preserving the natural physiological mobility, at least in part, of the vertebrae. Thus, these systems allow for procedures for effecting and maintaining, without osteosynthesis or graft for fusion, a correction of the relative positions of the vertebrae and/or of the forces exerted on the vertebrae for treating a congenital or acquired deformation of the spine, in particular an idiopathic deformation such as kypho-scoliosis, or a post-traumatic, tumorous, infectious, degenerative, or other instability of the spine, preserving at least in part the natural physiological mobility of the vertebrae.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A dynamic implanted spinal rod system, comprising
a first component comprising
a first proximal end having opposite flat upper and lower surfaces,
a first attachment opening passing from the upper surface to the lower surface,
a first movable insert disposed within the first attachment opening and having an insertion opening therein parallel to the first attachment opening to allow an anchoring component to be at least partially inserted therethrough, and
a distal end formed as a rod;
a second component comprising
a second proximal end having opposite flat upper and lower surfaces,
a second attachment opening disposed in the second proximal end passing from the upper surface to the lower surface,
a second movable insert disposed within the second attachment opening and having an insertion opening therein parallel to the second attachment opening to allow an anchoring component to be at least partially inserted therethrough, and
a distal end formed as a colleted tube for receiving the distal end of the first component.

2. The system of claim 1, wherein the first attachment opening comprises a sidewall having a ridge formed as a raised section of the sidewall encircling the entirety of the first attachment opening.

3. The system of claim 2, wherein the first movable insert comprises a notch formed around the periphery thereof, which resides upon the ridge in the first attachment opening.

4. The system of claim 1, wherein the first movable insert comprises a bio-compatible elastic polymer insert.

5. The system of claim 1, wherein the first proximal end comprises an enlarged body having an oval shape.

6. The system of claim 1, wherein the second attachment opening comprises a sidewall having a ridge formed as a raised section of the sidewall encircling the entirety of the second attachment opening.

7. The system of claim 6, wherein the second movable insert comprises a notch formed around the periphery thereof, which resides upon the ridge in the second attachment opening.

8. The system of claim 1, wherein the second movable insert comprises a bio-compatible elastic polymer insert.

9. The system of claim 1, wherein the second proximal end comprises an enlarged body having an oval shape.

10. The system of claim 1, wherein the second component further comprises a medial cylinder section between the second proximal end and the colleted tube.

11. The system of claim 1, wherein the distal end formed as a colleted tube for receiving the distal end of the first component comprises an open end at the distal end and at least one slot formed through a sidewall thereof.

12. The system of claim 1, wherein the distal end formed as a colleted tube comprises external threads formed on the tube for receiving an internally threaded nut.

13. The system of claim 1, further comprising an interconnector component comprising a distal end with a slot formed from the distal end thereof, running parallel to a long axis of the component, the upper and lower walls of the slot configured to interface around the upper and lower surfaces of the first proximal end, and an anchoring opening passing through the distal end, generally perpendicular to the slot and aligning with the first attachment opening, upon insertion of the first proximal end therein to allow an anchoring component to be placed therethrough.

14. A dynamic implanted spinal rod system, comprising
a proximal end having opposite flat upper and lower surfaces,
an attachment opening disposed in the proximal end passing from the upper surface to the lower surface,
a bio-compatible elastic polymer insert disposed within the attachment opening and having an insertion opening therein parallel to the attachment opening to allow an anchoring component to be at least partially inserted therethrough, and
a distal end formed as a colleted tube for receiving a cylindrical rod.

15. The system of claim 14, wherein the attachment opening comprises a sidewall having a step formed as a raised ridge on the sidewall parallel to the upper surface and lower surface.

16. The system of claim 15, wherein the bio-compatible elastic polymer insert comprises at least one notch formed within the periphery thereof, which resides upon the step in the attachment opening.

17. The system of claim 14, wherein the proximal end comprises an enlarged body having an oval shape.

18. The system of claim 14, wherein the system further comprises a medial cylinder section between the proximal end and the colleted tube.

19. The system of claim 14, wherein the distal end formed as a colleted tube for receiving a cylindrical rod comprises an open end at the distal end and at least one slot formed through a sidewall thereof.

20. The system of claim 14, wherein the distal end formed as a colleted tube comprises external threads formed on the tube for receiving an internally threaded nut.
